(19) United States
(12) Patent Application Publication
(10) Pub. No.: US 2024/0299435 A1
(43) Pub. Date: Sep. 12, 2024

(54) COMPOSITIONS CONTAINING ADENOSINE TRIPHOSPHATE (ATP) AND METHODS OF USE FOR COGNITIVE FUNCTION

(71) Applicant: TSI USA, LLC, Missoula, MT (US)

(72) Inventors: John RATHMACHER, Story City, IA (US); Larry KOLB, Missoula, MT (US); Shawn BAIER, Polk City, IA (US)

(21) Appl. No.: 18/573,609

(22) PCT Filed: Jun. 22, 2022
(86) PCT No.: PCT/US22/34510

§ 371 Date: Dec. 22, 2023

Related U.S. Application Data
(60) Provisional application No. 63/213,378, filed on Jun. 22, 2021.

(51) Int. Cl. A61K 31/7076; A61K 9/00; A61P 25/28

(52) U.S. Cl. CPC A61K 31/7076; A61K 9/0053; A61P 25/28

(57) ABSTRACT

Methods of use and compositions comprising a source of adenosine-5′-triphosphate (ATP) are provided. The administration of the compositions described improves cognitive function, reaction times, focus, mood, neuromuscular reactivity, and/or optimizes mental performance.

### CROSS REFERENCE TO RELATED APPLICATIONS

This application claims priority to International PCT Application No. PCT/US2022/034510, filed on Jun. 22, 2022, which claims priority to U.S. Provisional Application Ser. No. 63/213,378 filed on Jun. 22, 2021 and entitled “Compositions Containing Adenosine Triphosphate (ATP) and Methods of Use for Cognitive Function.” The entire contents of which are hereby expressly incorporated by reference in their entireties, including, without limitation, the specification, claims, and abstract, as well as any figures, tables, or drawings thereof.

### TECHNICAL FIELD

The present invention relates to a composition comprising adenosine-5′-triphosphate (ATP) and methods of using ATP to improve cognitive function, reaction times, focus, mood, neuromuscular reactivity, and/or to optimize mental performance.

### BACKGROUND

Adenosine-5′-triphosphate (ATP) has long been known as the chemical energy source for tissues including muscle. Intracellular ATP concentrations (1-10 mM) are quite high in contrast to extracellular concentrations (10-100 nM) and therefore release of ATP from cells such as erythrocytes and muscle is strictly controlled. More recently extracellular effects of ATP, acting through purinergic receptors found in most cell types, have been elicited. Several extracellular physiological functions of ATP have been described including vasodilation, reduced pain perception, and as a neurotransmission cotransmitter. Importantly, small and transient increases in vascular ATP in muscle can cause vasodilation and an increase in blood flow to the muscle.

Fatigue resistance in repeated high intensity bouts of exercise is a much sought-after attribute in athletics. This is true for both augmentation of training volume, as well as sustained force and power output in intermittent sports such as hockey. During fatiguing contractions acute adaptations in blood flow occur to stave off declines in force generating capacity. There is a tight coupling between oxygen demand in skeletal muscle and increases in blood flow. Research suggests that it is red blood cells that regulate this response by acting as “oxygen sensors”. ATP is carried in red blood cells and when oxygen is low in a working muscle region, the red blood cell deforms resulting in a cascade of events which lead to ATP release and binding to endothelial cells in smooth muscle. Binding results in smooth muscle relaxation and subsequent increases in blood flow, nutrient and oxygen delivery. Specifically, extracellular ATP directly promotes the increased synthesis and release of nitric oxide (NO) and prostacyclin (PGI₂) within skeletal muscle and therefore directly affects tissue vasodilation and blood flow. This is supported by research suggesting increased vasodilation and blood flow in response to intra-arterial infusion and exogenous administration of ATP. The outcome is maintenance of energy status in the cell under fatiguing contractions.

The physiological effects of ATP have led researchers to investigate the efficacy of oral supplementation of ATP. Jordan et al. demonstrated that 225 mg per day of enteric-coated ATP supplementation for 15 days resulted in increased total bench press lifting volume (i.e. sets.repetitions.load) as well as within-group set-one repetitions to failure. More recently, Rathmacher et al. found that 15 days of 400 mg per day of ATP supplementation increased minimum peak torque in set two of a knee extensor bout. ATP supplementation has previously demonstrated beneficial effects, including improved strength, power, and body composition. The effect of ATP supplementation on measures of cognition such as processing speed has not been investigated prior to the discovery of the present invention.

It has been unexpectedly and surprisingly discovered that ATP supplementation results in improved cognitive function, focus, mental performance, cognitive performance, mood, neuromuscular reactivity, and reaction time (RT). Further, ATP supplementation optimizes mental performance. Supplementation with ATP, especially at the levels described herein, does not appear to increase the body's total pool of ATP. Instead, these amounts of ATP increase signaling and blood flow, including blood flow to the brain and/or nutrient delivery.

Other objects, advantages and features of the present disclosure will become apparent from the following specification taken in conjunction with the accompanying figures.

### SUMMARY

One object of the present invention is to provide a composition and methods of use of the composition for use in improving cognitive function.

Another object of the present invention is to provide a composition and methods of use of the composition for improving focus.

A further object of the present invention is to provide a composition and methods of use of the composition for improving neuromuscular activity.

An additional object of the present invention is to provide a composition and methods of use of the composition for improving mood.

Another object of the present invention is to provide a composition and methods of use of the composition for improving reaction time.

A further object of the present invention is to provide a composition and methods of use of the composition for optimizing mental performance.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF THE FIGURES

FIG. 1A, FIG. 1B, and FIG. 1C show graphs of the results of the Dynavision Mode A hits testing. FIG. 1A=Changes within treatments across time (means±95% CI's); FIG. 1B=95% confidence intervals for within treatment changes between time points; FIG. 1C=Between treatment differences at 60 P (means±SD).

FIG. 2A, FIG. 2B, and FIG. 2C show graphs of the results of the Dynavision Mode A average reaction time (RT). FIG. 2A=Changes within treatments across time (means±95% CI's); FIG. 2B=95% confidence intervals for within treatment changes between time points; FIG. 2C=Between treatment differences at 60 P (means±SD).

FIG. 3A and FIG. 3B show graphs of the results of the Dynavision Mode B average reaction time (RT). Mode B avgRT. FIG. 3A=Changes within treatments across time (means±95% CI's); FIG. 3B=95% confidence intervals for within treatment changes between time points.

FIG. 4A and FIG. 4B show graphs of the results of the Dynavision Mode B misses. Mode B misses. FIG. 4A=Changes within treatments across time (means±95% CI's); FIG. 4B=95% confidence intervals for within treatment changes between time points.

### DETAILED DESCRIPTION

The present disclosure relates to the impact of adenosine-5′-triphosphate (ATP) supplementation on cognitive function, reaction time, mood, neuromuscular activity, and/or focus. The composition and methods of the present invention yield significant improvements in cognitive function, reaction time, mood, neuromuscular activity, and/or focus. The composition and methods are useful for optimizing mental performance.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the terms “adenosine triphosphate”, adenosine-5′-triphosphate and ATP is understood to refer to adenosine triphosphate, derivatives of adenosine triphosphate, analogs of adenosine triphosphate, and metabolites of adenosine triphosphate, unless otherwise indicated.

The embodiments of this disclosure are not limited to particular methods and compositions which can vary and are understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1%, and 4%. This applies regardless of the breadth of the range.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components, or ingredients, but only if the additional steps, components, or ingredients do not materially alter the basic and novel characteristics of the claimed methods and compositions.

Oral administration of ATP is usually in the form of Adenosine-5′-Triphospate Disodium. In the present invention, Adenosine-5′-Triphosphate Disodium or any form of ATP or adenosine suitable for oral administration may be combined with any of the known coatings suitable for imparting enteric properties in granular form.

One of skill in the art recognizes that ATP may be incorporated into the delivery and/or administration form in a fashion so as to result in a typical dosage range of about 10 mg to about 80 grams, though more or less may be desirable depending on the application and other ingredients. More specifically, a range of 200 mg to 500 mg per day is included in the present invention, including 200, 250, 300, 350, 400, 450, and 500 mg per day and every amount within this range.

The composition of ATP is administered to an animal in any suitable manner. Acceptable forms include, but are not limited to, solids, such as tablets or capsules, and liquids, such as enteral solutions. Also, the composition can be administered utilizing any pharmaceutically acceptable carrier. Pharmaceutically acceptable carriers are well known in the art and examples of such carriers include various starches and saline solutions. In the preferred embodiment, the composition is administered in an edible form. In addition, an effective dosage range may be administered in divided dosages, such as two to three times per day.

The present invention can be used with enteral feeding tubes that deliver nutrients and medications. Such feeding tubes may be used to deliver nutrients and medications to the stomach, small bowel, and jejunal regions. Feeding tubes may be nasoenteric, inserted through the mouth, or percutaneous. Enteral feeding may be administered by various methods, including continuous, cyclic, bolus and intermittent.

ATP is present in the composition in any form. A therapeutically effective range of ATP in the present invention includes ATP in the amount of around 10 milligrams to around 80 grams. In the preferred embodiment, the therapeutically effective range of ATP is around 100 milligrams to around 1.6 grams. More specifically, a range of 100 mg to 1600 mg per day is included in the present invention, including 100, 200, 300, 400, 500, 600, 700, 800, 900, 1000, 1100, 1200, 1300, 1400, 1500 and 1600 mg per day and every amount within this range.

When the composition is administered orally in an edible form, the composition is preferably in the form of a dietary supplement, foodstuff or pharmaceutical medium, more preferably in the form of a dietary supplement or foodstuff. Any suitable dietary supplement or foodstuff comprising the composition can be utilized within the context of the present invention. One of ordinary skill in the art will understand that the composition, regardless of the form (such as a dietary supplement, foodstuff or a pharmaceutical medium), may include amino acids, proteins, peptides, carbohydrates, fats, sugars, vitamins, phytochemicals, minerals and/or trace elements.

In order to prepare the composition as a dietary supplement or foodstuff, the composition will normally be combined or mixed in such a way that the composition is substantially uniformly distributed in the dietary supplement or foodstuff. Alternatively, the composition can be dissolved in a liquid, such as water, or emulsified in a liquid.

The composition of the dietary supplement may be a powder, a gel, a liquid or may be tabulated or encapsulated.

Although any suitable pharmaceutical medium comprising the composition can be utilized within the context of the present invention, preferably, the composition is combined with a suitable pharmaceutical carrier, such as dextrose or sucrose.

Methods of calculating the frequency by which the composition is administered are well-known in the art and any suitable frequency of administration can be used within the context of the present invention (e.g., one 400 mg dose per day or two 200 mg doses per day) and over any suitable time period (e.g., a single dose can be administered over a five-minute time period or over a one-hour time period, or, alternatively, multiple doses can be administered over an extended time period). The combination of ATP and nutritional materials (including nutrients, protein, peptides, vitamins, phytochemicals, minerals, fatty acids, and amino acids) and/or drugs can be administered over an extended period of time, such as weeks, months or years.

In some embodiments, the compositions may be delivered for a duration of about 3 days to about 365 days, about 5 days to about 365 days, about 10 days to about 365 days, about 14 days to about 365 days, about 21 days to about 365 days, about 3 days to about 100 days, about 5 days to about 60 days, about 7 days to 30 days, about 14 days to about 30 days, or about 21 days to 28 days. In some embodiments, the composition may be delivered for a duration of at least 21 days. In addition, without being limited according to the invention, all ranges recited are inclusive of the numbers defining the range and include each integer within the defined range.

Any therapeutically effective dose of ATP can be used within the context of the present invention. Methods of calculating proper doses are well known in the art. In certain embodiments, the compositions may be co-administered with an additional therapeutic agent.

The methods and compositions of the present invention can be administered to any person of any age, including healthy individuals, aging individuals, elderly individuals, individuals experiencing cognitive decline, individuals recovering from traumatic brain injury. In addition, the compositions and methods of the present invention can be used to improve cognitive function that has been impaired by fatigue, including fatigue in an individual experiencing fatigue from carrying out normal daily activities. By way of non-limiting example, an elderly person may experience fatigue that impacts cognitive function from simply performing normal daily activities.

The methods and compositions of the present invention can be used by individuals seeking optimization of mental performance, including but not limited to a person's mental performance relative to e-gaming, an athlete's ability to react faster or an individual seeking to stay focused during the work day.

### Experimental Examples

The effect of ATP on cognitive function, focus, mood and reaction time is studied in an exercise intervention model. Exercise intervention increases mood disturbance and elicits deficits in mood, reaction time and cognitive function. Administration of ATP modulates/attenuates mood disturbance, declines in reaction time and declines in cognitive performance as compared to a placebo. This attenuation occurs pre-exercise and post exercise. Exercise induces fatigue and results in a reduction in cognitive performance, mood, focus, and reaction time. ATP improves cognitive performance, mood and reaction time. It is well-known that reaction times are a measure of cognitive function in any individual, including healthy individuals, elderly individuals, individuals suffering from brain injury and/or individuals experiencing cognitive decline. The present invention is not limited to any particular type of individuals, nor is the present invention limited to exercising individuals. The experimental examples are non-limiting and one of skill in the art will recognize that the measures of cognitive function and cognitive performance are applicable to individuals of any age and fitness level. The examples described herein use models of fatigue to result in cognitive decline and/or impaired cognitive function. The results describe herein are applicable to all individuals, regardless of age, exercise status, or health.

### Study Design

This study followed a double-blind, randomized cross-over design. Participants were randomized to either PeakATP or Placebo and consumed their assigned supplement for a period of 14-days. Following supplementation, participants reported back to the lab within 24-hours for the first of two experimental trials, which occurred in a randomized, counterbalanced cross-over fashion. During experimental trial 1 (T1), participants ingested an acute dose of their assigned supplement 30 minutes before completing pre-testing assessments (Dynavision D2) followed by a 3-minute all-out high-intensity effort (3 MT) on a cycle ergometer. Participants repeated pre-testing assessments immediately (IP) and 60-minutes (60 P) post-completion of the 3 MT. Following completion of T1, participants underwent a 2-week wash-out period, followed by 14-days of supplementation with the supplement they did not consume during the first experimental trial (100 or 666). Participants returned to the study site within 24-hours of their last dose to complete experimental trial 2 (T2). Experimental trial 2 occurred in a fashion identical to T1, with acute ingestion of the supplement they did not consume during the first experimental trial occurring upon arrival to the lab (30 minutes before pre-testing).

### Participants

A convenience sample of healthy, recreationally active individuals were recruited. Thirty-five participants were enrolled in this study. Three participants were excluded due to failure to meet inclusion criteria, two were excluded for non-compliance and ten were lost to follow-up. The final analysis included twenty participants (10 men, 10 women) between the ages of 18 and 40 years old (22.3±4.4 yrs, 169.9±9.5 cm, 78.7±14.6 kg, 27.0±9.5% fat). To be included in this study, participants were required to be healthy and ready for activity as determined by physical activity readiness questionnaire (Par-Q+) and Medical History questionnaire (MHQ), be classified as recreationally active (≥150 minutes exercise per week) and not be taking and be willing to abstain from creatine or beta-alanine supplementation or be willing to complete a 4-week wash-out period prior to enrolling if taking creatine or beta-alanine and be willing to abstain from supplementing with either for the duration of the study.

### Warm-Up

Prior to the VO2 peak and 3 MT protocols, participants were required to complete a standardized dynamic warm-up. Participants completed 5-minutes of pedaling on a cycle ergometer at a resistance of 50 watts before completing ten body-weight squats, ten body-weight walking lunges, ten dynamic straight leg kicks, and ten dynamic walking quadricep stretches.

### VO2 Peak Test

All participants performed a ramp protocol to volitional exhaustion on a cycle ergometer (Lode, Excalibur Sport, Groningen, The Netherlands) to determine VO2 peak, peak power output (PPO) and power output at the gas exchange threshold (GET). Participants were instructed to maintain a pedaling cadence of 70-80 revolutions per minute (RPM) at an initial workload of 100 watts (W). The workload increased 30 W every two minutes (1 W per 2 seconds) until participants were unable to maintain a cadence above 70 RPM for ˜10 s despite verbal encouragement, or volitional fatigue. Expired gasses were analyzed using open-circuit spirometry (True One 2400® Metabolic Measurement System, Parvo-Medics Inc., Sandy, UT). VO2 peak (L·min⁻¹) was determined as the highest VO2 value achieved during the last completed stage of the test, coinciding with at least two of the following three parameters: heart rate (HR) within 10% of age-predicted maximal HR; respiratory exchange ratio (RER) of 1.15 or higher; a plateau in oxygen consumption despite an increase in exercise intensity. The highest power output achieved was recorded as peak power output (PPO) in watts (W). GET was determined via computerized regression analysis of the slopes of the CO2 uptake (VCO2) vs. O2 uptake (VO2). Power at the GET was recorded.

### Three Minute Test (3 MT)

The 3 MT is a 3-minute all-out high-intensity effort on a cycle ergometer (Lode, Excalibur Sport, Groningen, The Netherlands). Pilot testing for this study demonstrated that this protocol may elicit deficits in cognitive performance. After completing a standardized warm-up, participants completed 60 seconds of cycling (50 watts, 70-80 rpm) before immediately completing the 3 MT. Resistance during the test was set as a function of pedaling rate using a scaling factor based on the power output at a set cadence (70 rpm) being equal to 50% of the difference between the power output at GET and peak power output assessed during the VO2 peak test. To prevent pacing, participants were not informed of the elapsed time. The 3 MT was completed following PRE assessments during both experimental trial 1 and experimental trial 2.

### Assessments

### Dynavision Reaction Time Assessments

Reaction time was assessed using the Dynavision D2 visuomotor training device. The Dynavision D2 is a novel reaction time device developed to train sensory motor integration through the visual system. It consists of a 4 ft×4 ft computer integrated board with 64 tactile light emitting targets arranged into five concentric rings. During a test, illuminated targets serve as visual stimuli that require a physical hand strike to extinguish. The D2 can be programmed to create a number of RT assessments of variable frequency, duration and complexity that provide hit counts as well as visuomotor RT. It utilizes a large target field, which challenges both central and peripheral vision. Reaction time was assessed via two Dynavision D2 visuomotor tasks. The Dynavision D2 system is used to evaluate and train visual, cognitive and motor function across all ages, stages and conditions. It can be used to address underlying visual, cognitive and motor deficits including visual-motor reaction time, peripheral visual awareness, executive functions, active range-of-motion and dynamic balance. The Dynavision D2 system can be used to identify visual and cognitive defects after a brain injury, stroke or other neurologic pathology.

Mode A: The Mode A (proactive) task required participants to recognize and respond as fast as possible to random and sequentially appearing stimuli across the Dynavision apparatus target field. Following a 5-second visual countdown on the board's t-scope, an initial stimulus presented on the D2 board in a random location. The stimulus remained illuminated until the button was struck by the participant. The stimulus then changed to another random location. Participants were instructed to successfully identify and strike as many stimuli as possible within 60 seconds with both hands. The average of three discrete tests was utilized at each time point. The number of hits (hits) and average reaction time per hit (avgRT) were assessed.

Mode B: The Mode B (reactive) assessment is similar to Mode A. It required each participant to respond as fast as possible to random and sequentially appearing stimuli across the Dynavision apparatus target field. However, for the Mode B assessment the stimulus changed position to another random location within the Dynavision target field if not struck within 1 second. Additionally, participants were required to verbally recite a random 5-digit number that presents on the center screen (t-scope) of the D2 during each assessment. The randomly generated 5-digit number was presented a total of 11 times throughout the 60-second test, remaining for 0.75 seconds. The average of three discrete tests was utilized at each time point. The number of hits (hits), misses (misses) and the average reaction time per hit (avgRT) were assessed.

### Supplementation

Participants were assigned to consume either PeakATP (formula 100) or placebo (formula 666) for a 14-day period prior to completion of experimental trial 1 and experimental trial 2, which occurred in a randomized cross-over fashion. Participants also ingested an acute dose of the assigned supplement upon arrival to the lab at the beginning of experimental trials 1 and 2. PeakATP and placebo were obtained from TSI Group Ltd. (Missoula, MT, USA). Both the supplement and placebo were in the form of a flavored powder similar in taste and appearance provided in pre-portioned single serve stick packs. Each participant was provided with a 14-day supply of their assigned formula (PeakATP or placebo) following a familiarization session and experimental trial 1. Participants were instructed to mix their assigned formula in 8 oz of water and take 30 min before breakfast on an empty stomach.

Participants were required to keep a daily log detailing the date and time for which each dose is ingested. Participants were required to return all empty packets before beginning the experimental trials. Any remaining supplement was counted and recorded. During experimental trials 1 and 2, the supplement was taken immediately upon arrival to the lab 30 minutes prior to pre-testing. Supplementation compliance was 96.9% for Placebo (formula 666) and 98.6% for PeakATP (formula 100).

Peak ATP formula: 400 mg Peak ATP (adenosine 5′-triphosphate disodium), maltodextrin, silica-colloidal anhydrous, citric acid anhydrous, sucralose & guar gum.

Placebo Formula: Maltodextrin, silica-colloidal anhydrous, citric acid anhydrous, sucralose & guar gum.

### Statistical Analysis

A two-way (treatment [666 vs. 100]×time [PRE vs. IP vs. 60 P] repeated measures ANOVA was conducted to compare all dependent variables between treatments across time. If the assumption of sphericity is violated, a Greenhouse-Geisser correction was applied. Where there was a significant interaction, separate 1-way repeated-measures ANOVAs with Least Significant Difference (LSD) pairwise comparisons were used to assess changes in dependent variables for each treatment across time, with follow-up between treatment comparisons at each time point. All statistical procedures will be conducted using SPSS statistical software (v. 28.0.1.1) with a significance level set at p≤0.05.

### Results

### Dynavision Mode A

### Hits

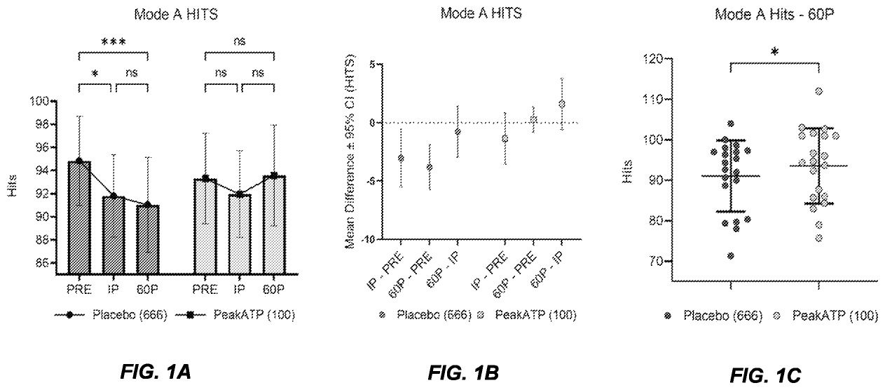

A significant time×treatment interaction was noted for number of hits (p=0.006). A significant time effect was noted in placebo (p=0.002), but not in ATP (p=0.187). In Placebo, the number of hits was significantly lower at IP (p=0.019) and 60 P (p<0.001) compared to PRE, whereas the number of hits was maintained in PeakATP. No differences were noted between treatments at PRE or IP (p′s>0.05). The number of hits was significantly greater in ATP at 60 P when compared to placebo (p=0.028). See FIGS. 1A, 1B, and 1C.

### AvqRT

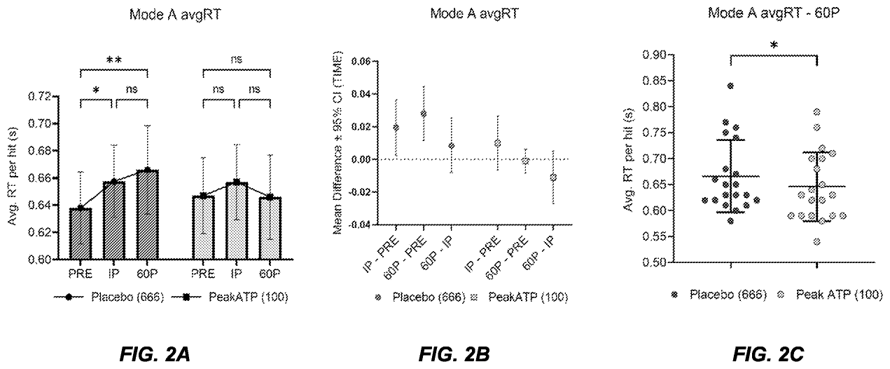

A significant time×treatment interaction was noted for avgRT (p=0.006). A significant time effect was noted in placebo (p=0.004), but not in PeakATP (p=0.211). In placebo, avgRT was significantly slower at IP (p=0.027) and 60 P (p=0.002) compared to PRE, whereas avgRT was maintained in PeakATP. No differences were noted between treatments at PRE or IP (p′s>0.05). avgRT was significantly faster in PeakATP at 60 P when compared to placebo (p=0.015). See FIGS. 2A, 2B, and 2C.

### Dynavision Mode B

### AvqRT

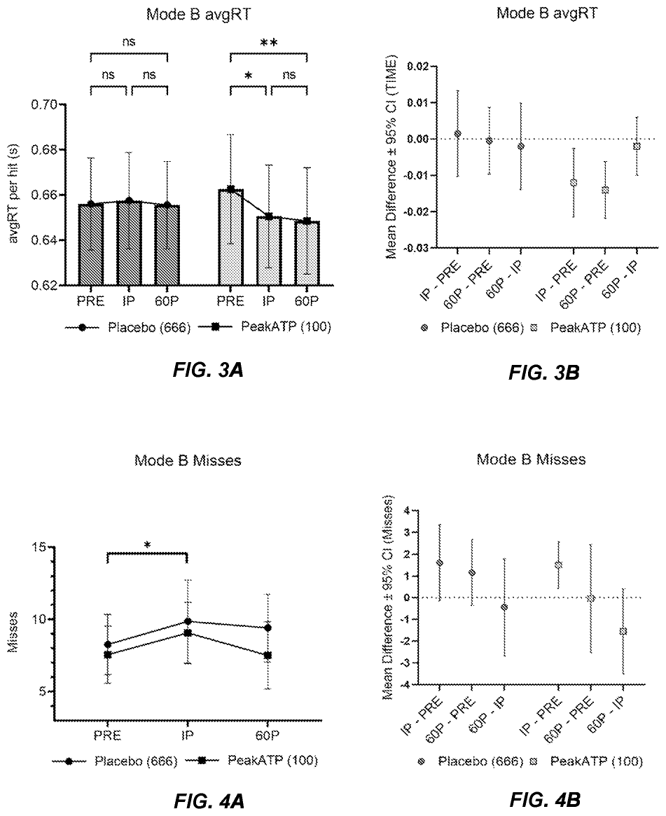

A significant time×treatment interaction was noted for avgRT (p=0.039). A significant time effect was noted in ATP (p=0.002), but not placebo (p=0.925). In ATP, avgRT was significantly faster at IP (p=0.015) and 60 P (p=0.001) compared to PRE. However, avgRT was not significantly different than placebo at any time point (p′s>0.05). See FIGS. 3A and 3B.

### Misses

No significant time×treatment interaction was noted for number of misses in Mode B. A significant main effect for time was noted (p=0.048) with the number of misses increasing at IP compared to PRE, regardless of treatment. A significant treatment effect was also noted (p=0.005) indicating that the number of misses was significantly lower in PeakATP overall when compared to placebo. See FIGS. 4A and 4B.

TABLE 1
Dynavision Data (means ± SD)

| Dynavision Task | Treatment | PRE | IP | 60P |
|---|---|---|---|---|
| Mode A Hits | PeakATP (100) | 93.30 ± 8.35 | 91.95 ± 8.00 | 93.57 ± 9.31 |
| | Placebo (666) | 94.83 ± 8.24 | 91.80 ± 7.65\hat{ } | 91.03 ± 8.80*^{§} |
| Mode A avgRT | PeakATP (100) | .647 ± .059 | .657 ± .059 | .646 ± .066 |
| | Placebo (666) | .638 ± .057 | .658 ± .057\hat{ } | .666 ± .069*^{§} |
| Mode B Hits | PeakATP (100) | 77.88 ± 11.14 | 79.13 ± 11.92 | 79.03 ± 12.03 |
| | Placebo (666) | 78.55 ± 11.01 | 77.37 ± 11.00 | 78.68 ± 10.54 |
| Mode B avgRT | PeakATP (100) | .663 ± .052 | .651 ± .049\hat{ } | .649 ± .050* |
| | Placebo (666) | .656 ± .043 | .658 ± .045 | .656 ± .041 |
| Mode B misses\hat{ }^{‡} | PeakATP (100) | 7.55 ± 4.21 | 9.05 ± 4.54 | 7.50 ± 4.99 |
| | Placebo (666) | 8.25 ± 4.44 | 9.85 ± 6.16 | 9.40 ± 5.01 |

\hat{ }IP Significantly different than PRE;
*60P Significantly different than PRE;
^{†} = 60P Significantly different than IP;
^{‡}significant treatment effect;
^{§}significantly difference between treatments

### DISCUSSION

Supplementation with ATP significantly attenuated the decline in the number of hits and average reaction time per hit in the Dynavision Mode A (proactive) reaction time assessment when compared to placebo. Average reaction time per hit was significantly slower and the number of hits was significantly lower immediately post-(IP) and 60-minutes post-exercise (60 P) when compared to PRE in placebo, whereas no significant declines were noted in ATP. Both the number of hits and average reaction time per hit were significantly better in ATP at 60 P compared to placebo. ATP supplementation prevents the decline in proactive visuomotor reaction time following all-out high-intensity exercise.

ATP significantly improved average reaction time (RT) per hit in the Dynavision Mode B (reactive) reaction time assessment, whereas no significant changes were noted in placebo. Average reaction time per hit was significantly faster immediately post-(IP) and 60-minutes post-exercise (60 P) when compared to PRE in ATP. ATP supplementation improves reactive visuomotor reaction time during a visuomotor task with cognitive stressor following all-out high-intensity exercise.

ATP significantly decreased the number of misses in the Dynavision Mode B (reactive) reaction time assessment across all time points when compared to placebo. ATP supplementation decreases the number of errors during a reactive visuomotor task with cognitive stressor before and after all-out high-intensity exercise.

ATP supplementation attenuates the decline in proactive visumotor RT, enhanced reactive visumotor RT and reduces the number of misses during the reactive visuomotor task.

The results demonstrate that the methods and compositions described herein provide for improvements in cognitive function, reaction time, mood, neuromuscular activity, and/or focus. In addition, the methods and compositions described herein provide for improvements in mental performance in an individual.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention. The terms subject and animal are used interchangeably throughout this application and are in no way limited to one term or the other.

1. A method of improving cognitive function in a person comprising administering to the person an effective amount of a composition comprising adenosine triphosphate (ATP), wherein the administration of the composition improves cognitive function.

2. The method of claim 1, wherein the person is administered a total daily dose of between about 100 mg and about 1600 mg of ATP.

3. The method of claim 1, wherein the composition is administered once daily.

4. The method of claim 1, wherein the composition is administered up to three times per day.

5. A method of improving focus in a person comprising administering to the person an effective amount of a composition comprising adenosine triphosphate (ATP), wherein the administration of the composition improves focus.

6. The method of claim 5, wherein the person is administered a total daily dose of between about 100 mg and about 1600 mg of ATP.

7. The method of claim 5, wherein the composition is administered once daily.

8. The method of claim 5, wherein the composition is administered up to three times per day.

9. A method of optimizing mental performance in a person comprising administering to the person an effective amount of a composition comprising adenosine triphosphate (ATP), wherein the administration of the composition optimizes mental performance.

10. The method of claim 9, wherein the person is administered a total daily dose of between about 100 mg and about 1600 mg of ATP.

11. The method of claim 9, wherein the composition is administered once daily.

12. The method of claim 9, wherein the composition is administered up to three times per day.

13. A method of improving reaction times in a person comprising administering to the person an effective amount of a composition comprising adenosine triphosphate (ATP), wherein the administration of the composition improves reaction times.

14. The method of claim 13, wherein the person is administered a total daily dose of between about 100 mg and about 1600 mg of ATP.

15. The method of claim 13, wherein the composition is administered once daily.

16. The method of claim 13, wherein the composition is administered up to three times per day.

17. An oral composition for improving cognitive function in an individual, comprising a source of adenosine triphosphate (ATP) and a pharmaceutically acceptable carrier.
